(19) United States
(12) Patent Application Publication
(10) Pub. No.: US 2018/0047065 A1
(43) Pub. Date: Feb. 15, 2018

(54) SYSTEM AND METHOD FOR PREDICTIVE DIGITAL PROFILES

(71) Applicant: ROYAL BANK OF CANADA, Montreal (CA)

(72) Inventor: Martin J. WILDBERGER, Toronto (CA)

(21) Appl. No.: 15/677,702

(22) Filed: Aug. 15, 2017

Related U.S. Application Data
(60) Provisional application No. 62/375,226, filed on Aug. 15, 2016.

(51) Int. Cl. G06Q 30/02; H04L 29/08; A61B 5/00; H04W 4/02

(52) U.S. Cl. CPC G06Q 30/0271; G06Q 30/0242; H04W 4/028; H04L 67/22; A61B 5/6801

(57) ABSTRACT

Computer implemented systems and methods for maintaining a digital profile configured for supporting automated prediction generation on a data storage unit are provided. The digital profile is periodically or continuously updated, and contains tracked information about a client (e.g., calendar information, physiological information, geographic location, financial transactional information), and information may be segmented such that select portions (e.g., approved via an opt-in) of the digital profile can be configured to be shared such that the client will be automatically exposed to contextual offers or coupons, in some situations. In other situations, the context may also automatically dictate that the client may not be receptive to contextual offers or coupons, and the system is configured to prevent the provisioning of contextual offers or coupons during specific time periods.

### CROSS-REFERENCED RELATED APPLICATIONS

The present application claims all benefit, including priority, to U.S. Provisional Application No. 62/375,226, entitled “SYSTEM AND METHOD FOR PREDICTIVE DIGITAL PROFILES” filed on Aug. 15, 2016, the content of which is fully incorporated herein.

### FIELD

The present disclosure generally relates to the field of electronically maintaining digital profiles, and more particularly, to systems, methods, and non-transitory computer readable media for maintaining predictive digital profiles.

### INTRODUCTION

Digital information pertaining to an client is useful when consolidated for analysis or prediction generation. Targeted offers and advertisements may provide improved outcomes when contextual factors are taken into consideration. Electronic information pertaining to the client, shared with the client's approval, may be useful for providing the contextual factors.

### SUMMARY

In various embodiments, computer implemented systems and methods configured to maintain a digital profile configured for supporting automated prediction generation on a data storage unit are provided. The digital profile is periodically or continuously updated, and contains tracked information about a client (e.g., calendar information, physiological information, geographic location, financial transactional information), and information may be segmented such that select portions (e.g., approved via an opt-in) of the digital profile can be configured to be shared such that the client will be automatically exposed to contextual offers or coupons, in some situations. In other situations, the context may also automatically dictate that the client may not be receptive to contextual offers or coupons, and the system is configured to prevent the provisioning of contextual offers or coupons during specific time periods.

A machine-implemented mechanism is utilized to automatically maintain and vary various aspects of the digital profile and to continuously or periodically process the digital profile to update identified patterns/trends based on tracked electronic information, provided in the form of electronic data sets. This information, for example, may be indicative of predictive patterns or predictive current states relating to the client's behaviors, moods, desires, among others.

In an embodiment, the system includes a digital footprint tracking engine (e.g., tracking a user's interaction, etc., with digital systems that leaves digital traces that can be acquired over time for use with dynamically updating the digital profile) configured to record an electronic approval for sharing of one or more portions of the digital profile and to receive electronic data sets containing client information associated with the client, each electronic data, encoded with timestamps, stored in the data storage unit, recorded into the digital profile, and assigned to a corresponding portion of the one or more portions of the digital profile based on an overall type of data in the electronic data set; a data encoding processor configured to extract, from the one or more portions of the digital profile approved for sharing, one or more data representations of the client's expected behavior, desires, or moods, the one or more data representations being appended onto the digital profile with a corresponding timestamp; a prediction engine configured to process the digital profile to identify one or more predictive relationships between the one or more data representations; and responsive to the identified one or more predictive relationships, to transmit a signal to cause the generation of a targeted advertisement or an targeted offer for provisioning to a device associated with the client.

In an embodiment, the one or more predictive relationships are generated at least based on temporal relationships identified between one or more representations having different corresponding timestamps.

In an embodiment, the one or more electronic data sets are automatically extracted from the device associated with the client, and the one or more electronic data sets include at least one of geospatial data, financial transaction history, mobile application usage, and sensory data.

In an embodiment, the sensory data is obtained from a wearable device associated with the client, and the wearable device is configured to track data representative of physiological signals associated with the client.

In an embodiment, the digital profile further includes a data structure indicating one or more types of targeted advertisements or one or more types of targeted offers that the client is willing to receive; and wherein the generated targeted advertisement or targeted offer is restricted to only a type of targeted advertisement or targeted offer indicated as acceptable in the data structure.

In an embodiment, the system is configured to maintain an overall score indicative of a quality of the digital profile, the overall score continuously updated in real or near-real time to reflect a perceived receptiveness of the client or the device associated with the client to the targeted advertisement or the targeted offer.

In an embodiment, the perceived receptiveness is varied at least by processing the one or more predictive relationships to identify temporal periods in which the client or the client device is likely to be in motion; and the perceived receptiveness is increased during the temporal periods in which the client or the client device is likely to be in motion.

In an embodiment, the perceived receptiveness is varied at least by processing the one or more predictive relationships to identify temporal periods in which the client or the client device is likely to be stationary; and the perceived receptiveness is decreased during the temporal periods in which the client or the client device is likely to be stationary.

In an embodiment, the one or more electronic data sets includes calendar information; the perceived receptiveness is varied at least by processing the one or more predictive relationships to identify temporal periods in which the client or the client device is likely to be in one or more meetings; and the perceived receptiveness is decreased during the temporal periods in which the client or the client device is likely to be in the one or more meetings.

In an embodiment, the provisioning of the targeted advertisement or the targeted offer occurs only when the overall score is greater than a pre-defined threshold.

In an embodiment, there is provided a computer implemented method for maintaining a digital profile configured for supporting automated prediction generation, the method including recording an electronic approval for sharing of one or more portions of the digital profile; receiving electronic data sets containing client information associated with the client, each electronic data set received, encoded with timestamps, stored in data storage, recorded into the digital profile, and assigned to a corresponding portion of the one or more portions of the digital profile based on an overall type of data in the electronic data set; processing the one or more portions of the digital profile approved for sharing to generate one or more data representations of the client's expected behavior, desires, or moods, the one or more data representations being appended onto the digital profile with a corresponding timestamp; processing the digital profile to identify one or more predictive relationships between the one or more data representations; responsive to the identified one or more predictive relationships, transmitting a signal to cause the generation of a targeted advertisement or an targeted offer for provisioning to a device associated with the client.

In accordance with one aspect, there is provided a system for maintaining one or more digital profiles, the system including a client data receiver configured to receive electronic data sets containing client information associated with a client, the electronic data sets received, encoded with timestamps, and stored in data storage; a predictor engine configured to, using at least the received electronic data sets containing the client information in combination with electronic data sets containing population-level information or context information, generate one or more behavioural predictions relating to the client, the one or more behavioural predictions including at least one or more quantified metrics; an advertising targeting engine that is configured to (i) generate one or more anonymized advertisement requests for transmission to an external advertising backend, the anonymized advertisement requests based at least on the one or more behavioural predictions, and (ii) receive one or more targeted advertisement requests from the external advertising backend, the one or more targeted advertisement requests indicative of electronic offers for provisioning to a computing device associated with the client; an advertisement hosting engine configured to receive the electronic offers and to control the computing device to cause the presentment of the electronic offers to the client; and a client profile management engine configured to maintain a client profile associated with the client, the client profile being stored in data storage and logging the electronic data sets containing the client information, the one or more behavioural predictions, and electronic records indicative of the electronic offers presented to the client through the advertisement hosting engine.

In accordance with another aspect, the client profile management engine is configured to maintain an opt-in trigger value stored indicative of the client's desire to receive the electronic offers through the client's computing device, the opt-in trigger value being used as a control input that determines whether the advertisement hosting engine presents the electronic offers to the client.

In accordance with another aspect, the client profile management engine is configured to maintain a data monetization trigger value stored indicative of the client's desire for sharing of a portion or all of the client's information stored on the client's profile with third party computing devices.

In accordance with another aspect, the client profile management engine is configured to monitor usage of the client's information or the portion thereof when used or traded by the third party computing devices.

In accordance with another aspect, the client profile management engine is configured to generate one or more rewards that are redeemable by the client based at least on the monitored usage of the client's information or the portion thereof.

In accordance with another aspect, the client profile management engine is configured for receiving client input controlling with which third parties associated with the external advertising backend the client profile is shared.

In accordance with another aspect, the client profile management engine is configured for receiving client input controlling what types of the electronic offers will be presented to the client through the client computing device.

In accordance with another aspect, the system further includes a loyalty determination engine configured to track the awarding and provision loyalty or other rewards for using the system, such provisioning triggered every time the client's information is shared, accessed, or acted upon.

In accordance with another aspect, the loyalty determination engine awards a quantity of loyalty or other rewards proportional to the proportion of the client's profile that the client has indicated, through one or more sharing flag values, an agreement to share with third parties associated with the external advertising backend.

In accordance with another aspect, the client profile management engine is configured to generate a profile quality score for the client, wherein a higher profile quality score is indicative that the client profile is more likely to be used by third parties.

In accordance with another aspect, the client profile score is adapted to vary in real-time throughout the course of the day to indicate the perceived receptiveness of the client to receiving communications at particular times.

In accordance with another aspect, the client data includes at least one of (i) client purchase history and future expected behavior (e.g., payments), (ii) information about any banking products or services used by the client, (iii) real-time or periodic data obtained from one or more wearable, mobile, or other computing devices used by the client; and (iv) any other internal or external data sources linked to the client.

In accordance with another aspect, the quantified metrics include quantified metrics associated with at least one of: (i) the client's expected behavior, (ii) the client's expected desires, and (iii) the client's expected moods.

In accordance with another aspect, the context information includes at least one of: (i) time of day, (ii) the client's location, (iii) the client's history/schedule, (iv) the client's tracked biometric information, and (v) the client's predicted routine.

In accordance with another aspect, the electronic offers are presented through at least one of: (i) an email, (ii) a text message, (iii) a companion application, (iv) a mobile wallet, and (v) a quick response code.

In accordance with another aspect, there is provided a method for maintaining one or more digital profiles, the method including: receiving electronic data sets containing client information associated with a client, the electronic data sets received, encoded with timestamps, and stored in data storage; generating one or more behavioural predictions relating to the client, the one or more behavioural predictions including at least one or more quantified metrics using at least the received electronic data sets containing the client information in combination with electronic data sets containing population-level information or context information; generating one or more anonymized advertisement requests for transmission to an external advertising backend, the anonymized advertisement requests based at least on the one or more behavioural predictions; receiving one or more targeted advertisement requests from the external advertising backend, the one or more targeted advertisement requests indicative of electronic offers for provisioning to a computing device associated with the client; receiving the electronic offers; controlling the computing device to cause the presentment of the electronic offers to the client; and maintaining a client profile associated with the client, the client profile being stored in data storage and logging the electronic data sets containing the client information, the one or more behavioural predictions, and electronic records indicative of the electronic offers presented to the client through an advertisement hosting engine.

In accordance with another aspect, there is provided a non-transitory computer-readable medium having instructions stored upon, the instructions, when executed, are configured to cause a processor to perform steps of a method including: receiving electronic data sets containing client information associated with a client, the electronic data sets received, encoded with timestamps, and stored in data storage; generating one or more behavioural predictions relating to the client, the one or more behavioural predictions including at least one or more quantified metrics using at least the received electronic data sets containing the client information in combination with electronic data sets containing population-level information or context information; generating one or more anonymized advertisement requests for transmission to an external advertising backend, the anonymized advertisement requests based at least on the one or more behavioural predictions; receiving one or more targeted advertisement requests from the external advertising backend, the one or more targeted advertisement requests indicative of electronic offers for provisioning to a computing device associated with the client; receiving the electronic offers; controlling the computing device to cause the presentment of the electronic offers to the client; and maintaining a client profile associated with the client, the client profile being stored in data storage and logging the electronic data sets containing the client information, the one or more behavioural predictions, and electronic records indicative of the electronic offers presented to the client through an advertisement hosting engine.

In various further aspects, the disclosure provides corresponding systems and devices, and logic structures such as machine-executable coded instruction sets for implementing such systems, devices, and methods.

In this respect, before explaining at least one embodiment in detail, it is to be understood that the embodiments are not limited in application to the details of construction and to the arrangements of the components set forth in the following description or illustrated in the drawings. Also, it is to be understood that the phraseology and terminology employed herein are for the purpose of description and should not be regarded as limiting.

Many further features and combinations thereof concerning embodiments described herein will appear to those skilled in the art following a reading of the instant disclosure.

### DESCRIPTION OF THE FIGURES

In the figures, embodiments are illustrated by way of example. It is to be expressly understood that the description and figures are only for the purpose of illustration and as an aid to understanding.

Embodiments will now be described, by way of example only, with reference to the attached figures, wherein in the figures:

FIG. 1 is a block schematic diagram illustrative of a system configured for providing predictive digital profiles, according to some embodiments;

FIG. 2 is a workflow diagram illustrative of a method for predictive digital profiles, according to some embodiments; and

FIG. 3 is a schematic diagram of computing device, exemplary of an embodiment.

### DETAILED DESCRIPTION

Embodiments of methods, systems, apparatus, or non-transitory computer readable media are described through reference to the drawings.

A computer-implemented system is provided that is configured for electronically maintaining digital profiles, and more particularly, for maintaining predictive digital profiles.

These digital profiles are adapted such that client data can be consolidated (or in some embodiments, transformed, interpolated, or extrapolated), and the digital profiles are operable for analysis or potential trading (in portions or in whole). In some embodiments, an opt-in trigger is available wherein clients are able to opt into having a portion or all of their data traded for other uses. A remuneration mechanism may be provided to compensate the client (e.g., by way of virtual tokens, payment of credits, contest submissions, rewards).

For example, where individuals have opted-in into information sharing, the system is configured to control the dissemination of their information while causing the triggering of control signals that instruct one or more computing systems to provision or generate rewards (e.g., virtual credits, offers, improved offers) to the client when the client's data is used or traded for particular purposes.

These control instructions, for example, may be generated electronic signals including instruction sets, machine code, or object code. Information on digital profiles may be stored in the form of data sets, for example, multi-dimensional vectors stored on a data storage, such as a relational database, a non-relational database, a flat file, among others. The digital profiles may be periodically or continuously maintained, and may include information that represents further processing (e.g., generated predictions, identified linkages, probabilistic relationships). The platform is configured to maintain a digital profile, and in some embodiments, is further configured for supporting automated prediction generation. The platform tracks a digital profile and maintains it such that opted-in clients may choose to have various portions of their digital profile sharable, or usable in generating aggregated information that is used for tailored or targeted advertisements. A potential benefit to the client is either payment in exchange for improved tailoring, and/or more relevant advertisements.

A client, for example, may include individuals such as members of the general public, users of a mobile application, individuals signed up on a mailing list, individuals having a pre-existing relation with an organization such as a non-profit, a retailer and a financial institution, individuals signed up on a loyalty program, among others. Client data may be provided from a variety of sources, including, for example: client purchase history and future expected behaviors (e.g., payments); information about any banking products or services used by the client; real-time or periodic data obtained from one or more wearable, mobile, or other computing devices used by the client; and any other internal or external data sources linked to the client (e.g. the client's calendar).

Digital information received or extracted from these sources may be processed and a digital profile may be created for each client by the system or a third party system with which the system is configured to distribute or share the digital profile (or portions thereof, and in some embodiments, anonymized). For example, a digital footprint tracking engine can be provided on one or more processors, configured to record an electronic approval for sharing of portions of the digital profile and to receive electronic data sets containing client information associated with the client. The digital footprint tracking engine is configured for tracking a user's interaction, etc., various digital systems that leave, for example, digital traces that can be acquired over time for use with dynamically updating the digital profile. These digital traces include transactions, physiological data, calendaring data, timestamped interactions (e.g., sent notes, emails), among others.

Electronic data can be encoded with timestamps, stored in a data storage unit (e.g., a database) and recorded into the digital profile. In some embodiments, the electronic data is classified and assigned to a corresponding portion of the digital profile based on an overall type of data in the electronic data set (e.g., event data may be saved as calendar data and categorized accordingly, as the client may only have opted-in to share or generate tailored predictions relating to calendar data).

The digital information may be utilized, for example, by the system or a third party system to predict the needs of the client in various circumstances, and the digital profile may contain some quantified metrics describing the client's expected behavior, desires, and moods, among others, and may use that information to target the client with communications or personalized ads at particular times. Predictions may be generated on a probabilistic basis, and may include contextual factors, such as time of day, season, weather, traffic conditions, etc. The contextual factors are prone to change, and accordingly, in some embodiments, the contextual factors are periodically or continuously monitored such that the system and its predictions are able to be responsive to just-in-time changes. In some embodiments, the platform includes a data encoding processor configured to extract, data representations (e.g., vectors, variables, scores) based on the client's expected behavior (e.g., going to the gym), desires (e.g., desires coffee in the morning), or moods (e.g., in a rush, angry), and these representations may be stored (e.g., appended) onto the digital profile with a corresponding timestamp.

Population-level data may be utilized in generating predictions that are then associated at the level of one or more individuals. The population-level data may be selected, for example, from individuals from similar demographics (e.g., with children), having similar lifestyle patterns (e.g., works from 9 AM-5 PM), or similar values (e.g., enjoys sports, likes coffee).

As an example, whether the client is sitting or walking/moving, the time of day, the client's location, the client's history/schedule, and predicted routine may be factored into communications pushed to the client through the client's mobile or wearable device. The system may computationally determine from information associated with the client's purchase history that the client typically makes a predictable purchase every day between 8:30 and 9:00 AM from one of a handful of coffee shops. In some embodiments, a prediction engine is provided that is configured to process the digital profile to identify relationships (e.g., patterns, trends) between the data representations. For example, a prediction engine may be configured to identify correlations, co-variances, linear relationships, non-linear relationships, lagged relationships (e.g., cyclical factors), that are indicative of potential sources of causation (e.g., if a meeting is booked in Mississauga, the client needs transportation) or inference (e.g., there is a 90% chance of a coffee purchase in the morning).

These relationships can be established by appending weighted linkages between different data elements, or comparing identified relationships against baselines defined by processing aggregate data from other data profiles and deviations therefrom (e.g., a consistent difference from a mean may be indicative of a trait of the user). A potential data structure includes representations of adjacency matrices, and weighted edge lists, whereby the adjacency matrices, and weighted edge lists are continuously updated to represent newly identified relationships.

Similarly, in some embodiments, deviations from normal patterns may be indicative of a change, or a potential opportunity (e.g., client is running late for work because of a subway malfunction as indicated by a geolocation status, whereby GPS data indicates that the person went from home to the subway, and back, and is still at home at 8 AM, when the client would normally be on the way to work). Accordingly, a data structure may include structured relationships and in some embodiments, represents a weighted directed graph where individual data points or abstractions thereof are assigned as nodes, and edge paths represent identified or probable relationships.

Based on these identified one or more predictive relationships, the system can transmit a control signal (e.g., to an advertisement network) to cause the generation of a targeted advertisement (e.g., based on the specific context or predicted context) or an targeted offer (e.g., a coupon) for provisioning to a device associated with the client. In some embodiments, the system rather includes a data set of appropriate advertisements along with contextual factors (e.g., only send on Tuesdays) and/or modification factors (e.g., add more discount factor if the client is identified by past redemption patterns to only incentivized by discounts over 25%). The consummation, interaction, or otherwise ignoring of various targeted advertisements or targeted offers can be tracked in the system or appended onto the digital profile such that improved tailoring is possible in the future. In some embodiments, an interface element is provided that allows for the tracking of responses (e.g., this restaurant offer is irrelevant, client indicates that client is a vegetarian).

The system may share that information with those or other coffee shops in the area, who may choose to act on that information to provide the client with a coupon or targeting advertising at or just before those times to go to a previously visited or new coffee shop. The coupon/ad may be pushed to the client's mobile or wearable device in an email, text message, or through a companion application, such as a mobile wallet. In the case of a coupon, the coupon could, for example, be provided as a quick response (QR) code or may be registered with the mobile wallet and automatically redeemed when the client makes a qualifying purchase using the mobile wallet.

In another example, the system may be configured to generate predictions when the client is trying to travel to work based on the client's schedule and location, or previous repeated daily behavior. If the client is delayed leaving the house and will be late for a meeting, or if a wearable device provides sensed information that the client is stressed (e.g., using heartrate or other sensors), the system may be configured to, using the digital profile in conjunction with the contextual information, target the client with a message suggesting the use of a taxi or other car service, while providing information for requesting a vehicle from the suggested service(s) through a single tap to the client's location.

The system might further be configured to provide a coupon for the use of such a service. In this specific example, the digital profile is continually updated with the client's physiological and geospatial coordinates, and combining that information with the calendar information, the digital profile includes one or more predictions of a desired location area. Responsive to real-time received information indicating that the client is potentially about to miss a meeting if an alternative form of transportation is not received, the system may generate a request to one or more advertisement partners to determine whether an offer is available to help the client consider alternative transportation (e.g., with a preferred carrier or company), such that the client obtains an improved offer that not only helps the client save money, but also helps the client arrive on time at the meeting.

Where the digital profile includes wearable information or other information for example, this information is utilized to tailor a request for a tailored advertisement or offer. For example, if wearable information includes data sets indicative of frantic movements (e.g., jerky geospatial movements), increased body temperature or increased heart rate, control signals sent to generate or otherwise provision advertisements may be modified accordingly (e.g., instead of a 20% discount, provide a 25% discount), etc.

The system may be configured for the distribution of information stored within the digital profile. As there may be privacy or other constraints, the system may be configured such that a field or a trigger is tracked to control with whom or with which systems the digital profile is shared (or how much of the digital profile is shared and if so, whether anonymizing is required to remove personal information), and what type of communications (e.g., medium, frequency, formatting) the client wishes to receive. In some embodiments, the digital profile dissemination and distribution configuration may be provisioned when the client initially registers for the system, and may be subsequently modified by the client, for example, through an application on a mobile computing device (e.g., a mobile wallet). The client may, for example, enter information in a digital form wherein the client may select (e.g., check off from a radio button list) which companies, or types of companies, may access the client's digital profile, and the client may select what kinds of information may be shared.

The system may be configured to provide loyalty or other rewards for using the system, such that every time the client's information is shared, accessed, or acted upon, the client may receive some reward, such as virtual points. Optionally, greater rewards may be offered to the client for agreeing to share more of the client's profile.

Through processing data from the variety of data sources, the system may be able to generate a score for each client, which may indicate the quality of the profile to prospective third parties. The higher the profile score, the more likely the profile is to be used by third parties. The score may vary in real-time throughout the course of the day to indicate the perceived receptiveness of the client to receiving communications at particular times. For example, it may not be helpful to target a client with communications when the client is busy in a meeting. The system might provide a reduced score for the user during those times, but a higher score when the user is on the move, away from the office.

The perceived receptiveness can be determined based on real- or near-real time data sets that indicate information that is proximate in temporal relevance, or part of an identified trend of potential future behaviors. For example, there may be a consistent Monday 9:30 AM meeting where the client is not receptive to offers or advertisements. Based on geolocation/geospatial data, calendar data, etc., the perceived receptiveness and accordingly, the overall quality score is reduced during these periods. Conversely, the perceived receptiveness can be increased afterwards when the client is determined to be moving after the meeting (e.g., GPS coordinate data indicates user is likely on the way to lunch).

Different vendors may choose to set coupons or other communications to be provisioned to clients having quality scores above a certain threshold only. The system may be automated, and accordingly, free of costly human tracking of quality scores. Similarly, when configuring the profile, the client may specify certain times in an interface (e.g., via interactive interface elements) to not receive any communications, or they may be a do not disturb feature, that the client may easily enable on the wearable or mobile device, preventing the system from targeting the client when that feature is active.

If the feature is active for several hours or more, the device might ask the client if the do not disturb (DND) setting should be cleared. The digital profile may be configured to track client settings, including, for example, indications related to whether the user would like frequent, a few, or no offers for a particular time period, such as a hour/day/week, etc.

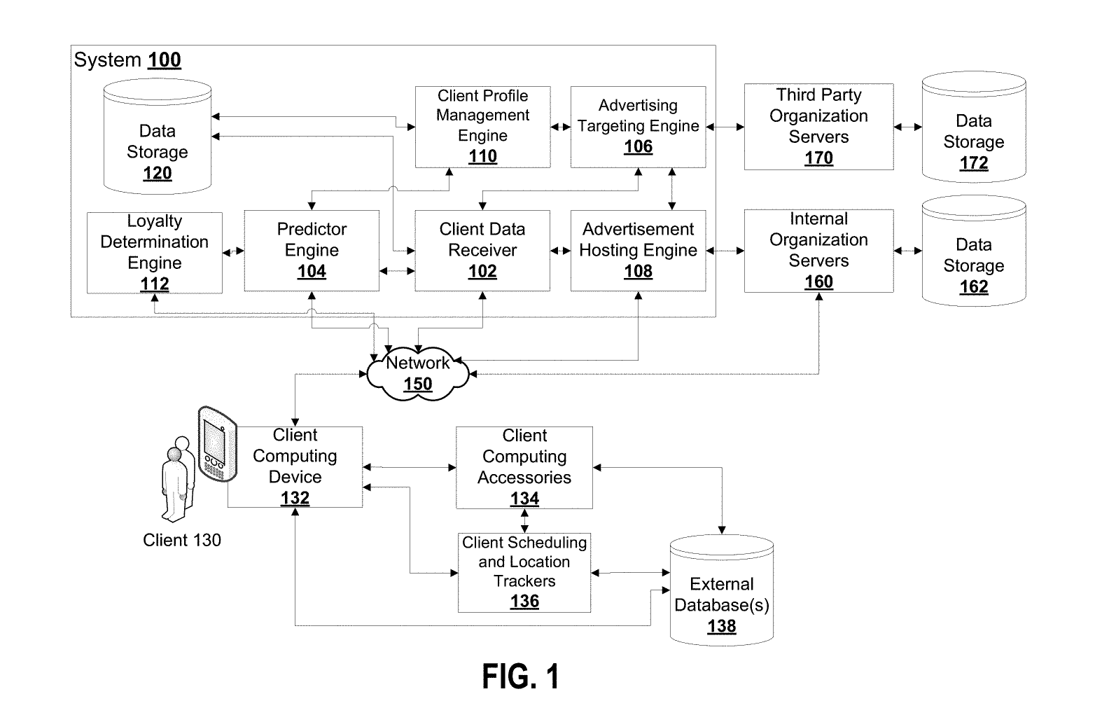

FIG. 1 is an example block schematic diagram illustrative of a system for providing predictive digital profiles, according to some embodiments.

System 100 may include, for example, client data receiver 102, predictor engine 104, advertising targeting engine 106, advertising hosting engine 108, and client profile management engine 110. The digital profile information and any received client data may be stored, for example, using data storage 120.

The system 100 may be in communication with a client 130 through client computing device 132, through, for example, network 150 (e.g., the Internet, an intranet, a wide area network, a local area network, a point-to-point connection). The system 100 may further be in electronic communication with an internal organization's servers 160 and their associated data storage 162. The internal organization's servers 160 and their associated data storage 162 may be associated with an organization that is directly linked to the system 100, such as a financial institution that provides system 100 as a service to its banking clients. The system 100 may further be in electronic communication with third party organization servers 170 and their associated data storage 172.

The system 100 is configured for, over a period of time or based on a sufficiently large initial set of data, maintaining digital profiles for each client based potentially on data obtained, determined, interpolated, or extrapolated about each client based on data retrieved from a variety of sources. Other components, modules, or blocks are possible. System 100 may be an architecture, a digital provisioning infrastructure, a software platform, a hardware platform, among others. In some embodiments, a special purpose machine is provided wherein components are specifically selected and configured to perform a limited range of functions efficiently, such as an application specific integrated circuit. System 100 is illustrated as an example, and there may be alternate, different, more, or less components, modules, or blocks.

The client data receiver 102 may part of a digital footprint tracking engine that maintains the digital profile, and causes the invocation of data record updating functions responsive to received information. Client data receiver 102 is configured to receive electronic data sets containing client information associated with a client, the electronic data sets received, encoded with timestamps, and stored in data storage 120. These data sets may be provided in the form of electronic records, streams of electronic data, etc.

The client data receiver 102 may receive the electronic data sets through client computing device 132 and the electronic data sets may further include electronic information relating to the client communicated through client computing device 132 or received directly from data sources, such as information obtained from client computing accessories 134 (which, for example, may include wearable devices, Internet of Things enabled appliances, etc.), client scheduling and location trackers 136 (e.g., calendar information, location information, movement information, gyroscopic information, accelerometer information, proximity sensors, among others), and external databases 138 (e.g., facility entry/exit logs at a workplace or a recreational facility).

In some embodiments, the client data includes at least one of client purchase history and future expected payments, information about any banking products or services used by the client, real-time or periodic data obtained from one or more wearable, mobile, or other computing devices used by the client; and any other internal or external data sources linked to the client. A data encoding processor is provided to convert the received electronic data sets into portions of a digital profile, and timestamp the information for downstream processing and modification. The data, in some embodiments, is transformed and/or compressed to improve ease of processing (e.g., simplifications, removal of extraneous data points, aggregation of similar data).

Predictor engine 104 is configured to, using at least the received electronic data sets containing the client information in combination with electronic data sets containing population-level information or context information, generate one or more behavioural predictions relating to the client, the one or more behavioural predictions including at least one or more quantified metrics. The predictor engine 104, in some embodiments, is configured to first identify an anchor set of baseline predictions about a client, stored in the form of nodes of a directed graph. The directed graph may then be updated with linkages (e.g., appended) and re-weighted as data is received about the digital profile, and interactions with advertisements/offers provided by the system.

For example, predictions may be generated through the use of weighted comparisons to identify differences or similarities between historical patterns for the individual, identified population-level patterns (e.g., for the general population as a whole or a selected demographic segment), among others. Quantified metrics may be utilized to generate predictions, including quantified metrics associated with at least one of: the client's expected behavior, the client's expected desires, and the client's expected moods. Machine-learning, trained neural networks, and hidden Markov models, among others, can be utilized to identify relationships and weightings thereof based on a sufficiently large or trained data set.

In some embodiments, predictor engine 104 applies one or more predictive models wherein context information is provided into the predictor engine 104 that aids in tailoring predictions based on known context. Context may include, for example, known information regarding timing, scheduling, appetite, real-world events that may impact the validity and reliability of a prediction that is otherwise devoid of context. In some embodiments, the context information includes at least one of: (i) time of day, (ii) the client's location, (iii) the client's history/schedule, (iv) the client's tracked biometric information, and (v) the client's predicted routine.

The predictor engine 104 may be utilized to proactively estimate future state information associated with a client such that targeted advertisements may be adapted so that the advertisements have greater relevance to the client, for example, aiding the client in decision making, etc. These predictions may be classified behavioural predictions, and may have one or more timestamps or metadata indicating the period of validity of said predictions in view of contextual or other modification factors.

The predictions may be generated using, for example, comparisons with predictive models wherein a level of similarity or correspondence is developed having a particular confidence level or score associated with the prediction. In some embodiments, the predictor engine 104 is configured to only select those predictions having a confidence level or score higher than a pre-determined threshold.

Advertising targeting engine 106 is configured to (i) generate one or more anonymized advertisement requests for transmission to an external advertising backend, the anonymized advertisement requests based at least on the one or more behavioural predictions, and (ii) receive one or more targeted advertisement requests from the external advertising backend (e.g., third party organizational servers 170 and associated data storage 172), the one or more targeted advertisement requests indicative of electronic offers for provisioning to a computing device associated with the client.

The anonymized advertisement requests may be adapted to remove or redact identifying information prior to transmission. Advertisement hosting engine 108 is configured to receive the electronic offers and to control the computing device to cause the presentment of the electronic offers to the client.

Client profile management engine 110 is configured to maintain a client profile associated with the client, the client profile being stored in data storage 120 and logging the electronic data sets containing the client information, the one or more behavioural predictions, and electronic records indicative of the electronic offers presented to the client through the advertisement hosting engine 108.

In some embodiments, the client profile management engine 110 is configured to maintain an opt-in trigger value stored indicative of the client's desire to receive the electronic offers through the client's computing device, the opt-in trigger value being used as a control input that determines whether the advertisement hosting engine 108 presents the electronic offers to the client.

In some embodiments, the client profile management engine 110 is configured to maintain a data monetization trigger value stored indicative of the client's desire for sharing of a portion or all of the client's information stored on the client's profile with third party computing devices. In some embodiments, the client profile management engine 110 is configured to monitor usage of the client's information or the portion thereof when used or traded by the third party computing devices.

In some embodiments, the client profile management engine 110 is configured to generate one or more rewards that are redeemable by the client based at least on the monitored usage of the client's information or the portion thereof. In some embodiments, the client profile management engine 110 is configured for receiving client input controlling with which third parties associated with the external advertising backend the client profile is shared.

In some embodiments, the client profile management engine 110 is configured for receiving client input controlling what types of the electronic offers will be presented to the client through the client computing device.

In some embodiments, the system 100 further comprising a loyalty determination engine 112 configured to track the awarding and provision loyalty or other rewards for using the system, such provisioning triggered every time the client's information is shared, accessed, or acted upon.

In some embodiments, the loyalty determination engine 112 is configured to award a quantity of loyalty or other rewards proportional to the proportion of the client's profile that the client has indicated, through one or more sharing flag values, an agreement to share with third parties associated with the external advertising backend.

In some embodiments, the client profile management engine 110 is configured to generate a profile quality score for the client, wherein a higher profile quality score is indicative that the client profile is more likely to be used by third parties. In some embodiments, wherein the client profile score is adapted to vary in real-time throughout the course of the day to indicate the perceived receptiveness of the client to receiving communications at particular times.

In some embodiments, the electronic offers are presented through at least one of: (i) an email, (ii) a text message, (iii) a companion application, (iv) a mobile wallet, and (v) a quick response code.

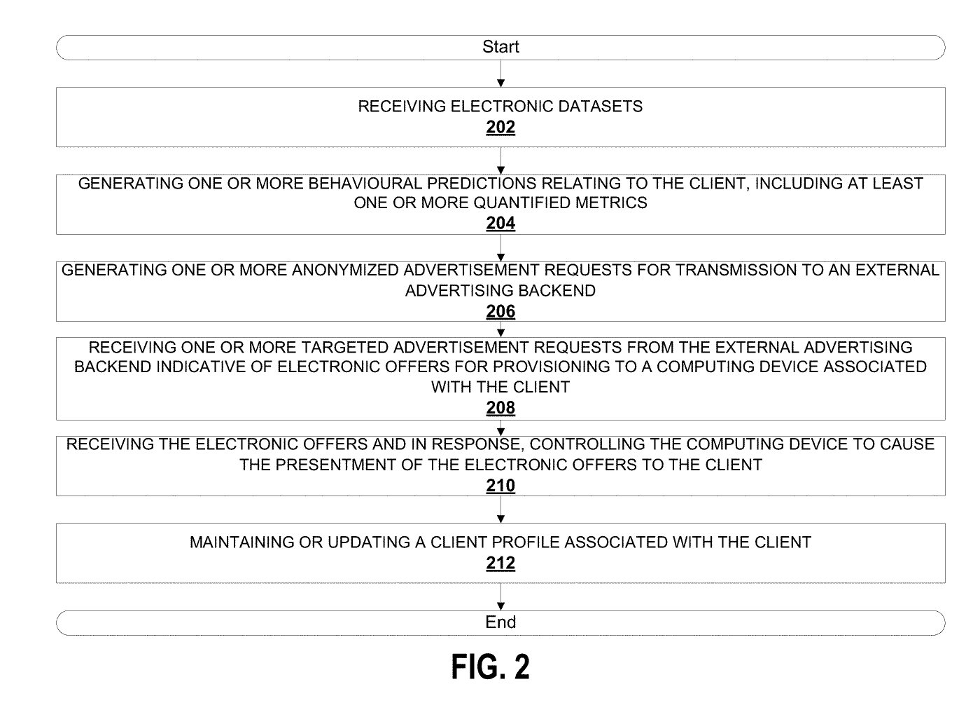

At FIG. 2, an example workflow is illustrated for maintaining one or more digital profiles, according to some embodiments.

The method 200 is illustrated and may include the following steps. The steps are illustrated as examples and there may be more, less, alternate, or different steps. The steps may be performed in various orders and the order shown is not limiting. The steps may also be combined together or separated apart as separate sub-steps.

At 202, a device may be instructed for receiving electronic data sets containing client information associated with a client, the electronic data sets received, encoded with timestamps, and stored in data storage.

At 204, the device may be instructed for generating one or more behavioural predictions relating to the client, the one or more behavioural predictions including at least one or more quantified metrics using at least the received electronic data sets containing the client information in combination with electronic data sets containing population-level information or context information.

At 206, the device may be instructed for generating one or more anonymized advertisement requests for transmission to an external advertising backend, the anonymized advertisement requests based at least on the one or more behavioural predictions.

At 208, the device may be instructed for receiving one or more targeted advertisement requests from the external advertising backend, the one or more targeted advertisement requests indicative of electronic offers for provisioning to a computing device associated with the client.

At 210, the device may be instructed for receiving the electronic offers and in response, controlling the computing device to cause the presentment of the electronic offers to the client.

At 212, the device may be instructed for maintaining or updating a client profile associated with the client, the client profile being stored in data storage and logging the electronic data sets containing the client information, the one or more behavioural predictions, and electronic records indicative of the electronic offers presented to the client through an advertisement hosting engine.

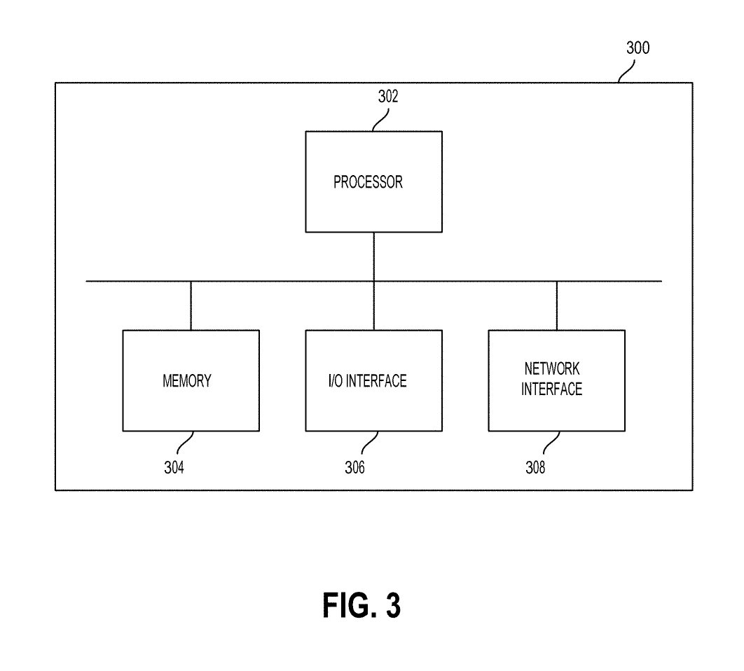

FIG. 3 is a schematic diagram of computing device 300, exemplary of an embodiment. As depicted, computing device includes at least one processor 302, memory 304, at least one I/O interface 306, and at least one network interface 308.

Processor 302 may be an Intel or AMD x86 or x64, PowerPC, ARM processor, among others. Memory 304 may include a combination of computer memory that is located either internally or externally such as, for example, random-access memory (RAM), read-only memory (ROM), compact disc read-only memory (CDROM), electro-optical memory, magneto-optical memory, erasable programmable read-only memory (EPROM), and electrically-erasable programmable read-only memory (EEPROM), Ferroelectric RAM (FRAM) or the like.

Each I/O interface 306 enables computing device 300 to interconnect with one or more input devices, such as a keyboard, mouse, camera, touch screen and a microphone, or with one or more output devices such as a display screen and a speaker.

Each network interface 308 enables computing device 300 to communicate with other components, to exchange data with other components, to access and connect to network resources, to serve applications, and perform other computing applications by connecting to a network (or multiple networks) capable of carrying data including the Internet, Ethernet, plain old telephone service (POTS) line, public switch telephone network (PSTN), integrated services digital network (ISDN), digital subscriber line (DSL), coaxial cable, fiber optics, satellite, mobile, wireless (e.g. Wi-Fi, WMAX), SS7 signaling network, fixed line, local area network, wide area network, and others, including any combination of these.

Computing device 300 is operable to register and authenticate users (using a login, unique identifier, and password for example) prior to providing access to applications, a local network, network resources, other networks and network security devices. Computing devices 300 may serve one user or multiple users.

The embodiments of the devices, systems and methods described herein may be implemented in a combination of both hardware and software. These embodiments may be implemented on programmable computers, each computer including at least one processor, a data storage system (including volatile memory or non-volatile memory or other data storage elements or a combination thereof), and at least one communication interface.

Program code is applied to input data to perform the functions described herein and to generate output information. The output information is applied to one or more output devices. In some embodiments, the communication interface may be a network communication interface. In embodiments in which elements may be combined, the communication interface may be a software communication interface, such as those for inter-process communication. In still other embodiments, there may be a combination of communication interfaces implemented as hardware, software, and combination thereof.

Throughout the foregoing discussion, numerous references will be made regarding servers, services, interfaces, portals, platforms, or other systems formed from computing devices. It should be appreciated that the use of such terms is deemed to represent one or more computing devices having at least one processor configured to execute software instructions stored on a computer readable tangible, non-transitory medium. For example, a server can include one or more computers operating as a web server, database server, or other type of computer server in a manner to fulfill described roles, responsibilities, or functions.

Although the embodiments have been described in detail, it should be understood that various changes, substitutions and alterations can be made herein without departing from the scope of various embodiments.

Moreover, the scope of the present application is not intended to be limited to the particular embodiments of the process, machine, manufacture, composition of matter, means, methods and steps described in the specification. As one of ordinary skill in the art will readily appreciate from the disclosure, processes, machines, manufacture, compositions of matter, means, methods, or steps, presently existing or later to be developed, that perform substantially the same function or achieve substantially the same result as the corresponding embodiments described herein may be utilized. Accordingly, the appended claims are intended to include within their scope such processes, machines, manufacture, compositions of matter, means, methods, or steps.

As can be understood, the examples described above and illustrated are intended to be exemplary only.

What is claimed is:

1. A computer implemented system configured to maintain a digital profile configured for supporting automated prediction generation on a data storage unit, the system comprising:
a digital footprint tracking engine configured to record an electronic approval for sharing of one or more portions of the digital profile and to receive electronic data sets containing client information associated with the client, each electronic data, encoded with timestamps, stored in the data storage unit, recorded into the digital profile, and assigned to a corresponding portion of the one or more portions of the digital profile based on an overall type of data in the electronic data set;
a data encoding processor configured to extract, from the one or more portions of the digital profile approved for sharing, one or more data representations of the client's expected behavior, desires, or moods, the one or more data representations being appended onto the digital profile with a corresponding timestamp;
a prediction engine configured to process the digital profile to identify one or more predictive relationships between the one or more data representations; and
responsive to the identified one or more predictive relationships, to transmit a signal to cause the generation of a targeted advertisement or an targeted offer for provisioning to a device associated with the client.

2. The computer implemented system of claim 1, wherein the one or more predictive relationships are generated at least based on temporal relationships identified between one or more representations having different corresponding timestamps.

3. The computer implemented system of claim 1, wherein the one or more electronic data sets are automatically extracted from the device associated with the client, and the one or more electronic data sets include at least one of geospatial data, financial transaction history, mobile application usage, and sensory data.

4. The computer implemented system of claim 3, wherein the sensory data is obtained from a wearable device associated with the client, and the wearable device is configured to track data representative of physiological signals associated with the client.

5. The computer implemented system of claim 1, wherein the digital profile further includes a data structure indicating one or more types of targeted advertisements or one or more types of targeted offers that the client is willing to receive; and wherein the generated targeted advertisement or targeted offer is restricted to only a type of targeted advertisement or targeted offer indicated as acceptable in the data structure.

6. The computer implemented system of claim 1, wherein the prediction engine is configured to maintain an overall score indicative of a quality of the digital profile, the overall score continuously updated in real or near-real time to reflect a perceived receptiveness of the client or the device associated with the client to the targeted advertisement or the targeted offer.

7. The computer implemented system of claim 6, wherein the perceived receptiveness is varied at least by processing the one or more predictive relationships to identify temporal periods in which the client or the client device is likely to be in motion; and
wherein the perceived receptiveness is increased during the temporal periods in which the client or the client device is likely to be in motion.

8. The computer implemented system of claim 6, wherein the perceived receptiveness is varied at least by processing the one or more predictive relationships to identify temporal periods in which the client or the client device is likely to be stationary; and
wherein the perceived receptiveness is decreased during the temporal periods in which the client or the client device is likely to be stationary.

9. The computer implemented system of claim 6, wherein the one or more electronic data sets includes calendar information;
wherein the perceived receptiveness is varied at least by processing the one or more predictive relationships to identify temporal periods in which the client or the client device is likely to be in one or more meetings; and
wherein the perceived receptiveness is decreased during the temporal periods in which the client or the client device is likely to be in the one or more meetings.

10. The computer implemented system of claim 6, wherein the provisioning of the targeted advertisement or the targeted offer occurs only when the overall score is greater than a pre-defined threshold.

11. A computer implemented method for maintaining a digital profile configured for supporting automated prediction generation, the method comprising:
recording an electronic approval for sharing of one or more portions of the digital profile;
receiving electronic data sets containing client information associated with the client, each electronic data set received, encoded with timestamps, stored in data storage, recorded into the digital profile, and assigned to a corresponding portion of the one or more portions of the digital profile based on an overall type of data in the electronic data set;
processing the one or more portions of the digital profile approved for sharing to generate one or more data representations of the client's expected behavior, desires, or moods, the one or more data representations being appended onto the digital profile with a corresponding timestamp;
processing the digital profile to identify one or more predictive relationships between the one or more data representations;
responsive to the identified one or more predictive relationships, transmitting a signal to cause the generation of a targeted advertisement or an targeted offer for provisioning to a device associated with the client.

12. The computer implemented method of claim 11, wherein the one or more predictive relationships are generated at least based on temporal relationships identified between one or more representations having different corresponding timestamps.

13. The computer implemented method of claim 11, wherein the one or more electronic data sets are automatically extracted from the device associated with the client, and the one or more electronic data sets include at least one of geospatial data, financial transaction history, mobile application usage, and sensory data.

14. The computer implemented method of claim 13, wherein the sensory data is obtained from a wearable device associated with the client, and the wearable device is configured to track data representative of physiological signals associated with the client.

15. The computer implemented method of claim 11, wherein the digital profile further includes a data structure indicating one or more types of targeted advertisements or one or more types of targeted offers that the client is willing to receive; and wherein the generated targeted advertisement or targeted offer is restricted to only a type of targeted advertisement or targeted offer indicated as acceptable in the data structure.

16. The computer implemented method of claim 11, comprising maintaining an overall score indicative of a quality of the digital profile, the overall score continuously updated in real or near-real time to reflect a perceived receptiveness of the client or the device associated with the client to the targeted advertisement or the targeted offer.

17. The computer implemented method of claim 16, wherein the perceived receptiveness is varied at least by processing the one or more predictive relationships to identify temporal periods in which the client or the client device is likely to be in motion; and
wherein the perceived receptiveness is increased during the temporal periods in which the client or the client device is likely to be in motion.

18. The computer implemented method of claim 16, wherein the perceived receptiveness is varied at least by processing the one or more predictive relationships to identify temporal periods in which the client or the client device is likely to be stationary; and
wherein the perceived receptiveness is decreased during the temporal periods in which the client or the client device is likely to be stationary.

19. The computer implemented method of claim 16, wherein the one or more electronic data sets includes calendar information;
the perceived receptiveness is varied at least by processing the one or more predictive relationships to identify temporal periods in which the client or the client device is likely to be in one or more meetings; and
wherein the perceived receptiveness is decreased during the temporal periods in which the client or the client device is likely to be in the one or more meetings.

20. A computer readable medium including machine readable instructions for maintaining a digital profile configured for supporting automated prediction generation, the machine readable instruction, when executed by a processor, cause the processor to perform a method comprising:
recording an electronic approval for sharing of one or more portions of the digital profile;
receiving electronic data sets containing client information associated with the client, each electronic data set received, encoded with timestamps, stored in data storage, recorded into the digital profile, and assigned to a corresponding portion of the one or more portions of the digital profile based on an overall type of data in the electronic data set;
processing the one or more portions of the digital profile approved for sharing to generate one or more data representations of the client's expected behavior, desires, or moods, the one or more data representations being appended onto the digital profile with a corresponding timestamp;
processing the digital profile to identify one or more predictive relationships between the one or more data representations;
responsive to the identified one or more predictive relationships, transmitting a signal to cause the generation of a targeted advertisement or an targeted offer for provisioning to a device associated with the client.
